(19) United States
(12) Patent Application Publication
(10) Pub. No.: US 2023/0067388 A1
(43) Pub. Date: Mar. 2, 2023

(54) SYSTEM FOR A DYNAMIC ELECTRICALLY STIMULATING ROD-LIKE ORTHOPEDIC IMPLANT

(71) Applicant: Intelligent Implants Limited, Cork (IE)

(72) Inventors: Erik Robert Zellmer, Gothenburg (SE); John Michael Zellmer, Gothenburg (SE)

(21) Appl. No.: 17/894,683

(22) Filed: Aug. 24, 2022

Related U.S. Application Data
(60) Provisional application No. 63/236,641, filed on Aug. 24, 2021.

(51) Int. Cl. A61N 1/378; A61B 5/00

(52) U.S. Cl. CPC A61N 1/378; A61B 5/0031; A61N 1/04

(57) ABSTRACT

A system for an electrically stimulating orthopedic implant includes: a rod-like orthopedic implant, comprising a shaft of the orthopedic implant, with distinct electrode sites situated along the implant body; and an end, comprising the head of the orthopedic implant, situated at one end of the shaft; a set of electrodes, individually controllable, wherein each electrode: includes a distinct stimulation site, comprising an active exposed segment of the electrode situated on an electrode site, is conductively coupled to an implant control circuitry, and is conductively isolated from all other electrodes in the set of electrodes; and implant circuitry, situated at least partially within the end cap, comprising: implant receiver circuitry, effective to convert an electromagnetic field to an electric current and implant control circuitry, configured to control current flow through the set of electrodes.

### CROSS-REFERENCE TO RELATED APPLICATIONS

This application claims the benefit of U.S. Provisional Application No. 63/236,641, filed on 24 Aug. 2021, which is incorporated in its entirety by this reference.

### TECHNICAL FIELD

This invention relates generally to the field of orthopedic implants, and more specifically to a new and useful system for a dynamic electrically stimulating orthopedic implant.

### BACKGROUND

Orthopedic surgery is one of the most common branches of surgery performed within the US and in Europe, where orthopedic surgeons use many means to treat musculoskeletal trauma, spine diseases, sport injuries, degenerative diseases, infections, tumors and congenital disorders. Very often, the goal of orthopedic surgery is to introduce, replace, or connect musculoskeletal tissue. An intermedullary rod (also known as intramedullary nail or inter-locking nail, or simply surgical nail) is a metal rod forced into the medullary cavity of a bone. Surgical nails have long been used to treat fractures and have more recently been applied to a wider range of orthopedic injuries. With the broad usage of surgical nails, there is an impetus to enable the surgical nail, and other orthopedic implants, to have enhanced functionalities to improve tissue growth and to enable better and additional patient telemetry/monitoring.

As the technology and usage of surgical nail hardware improves, and more generally, the technology and usage of long-term implants improve, a greater emphasis needs to be placed on placement and preservation of electronic components of the implant. With improving technologies, electronic components play a greater role in the effectiveness of the implant but are often the most susceptible to deterioration over time within the body and may additionally cause the most harm to the body if they start breaking down or the device becomes damaged. Furthermore, many of the materials and manufacturing techniques previously used in medical devices suffer from incompatibility with electronics. For example, manufacturing temperatures for some materials such as PEEK (polyether ether ketone) could destroy adjacent electronics. Thus, there is a need in the medical implant field to create a new and useful system for embedding electronic components within an implant. This invention provides such a new and useful system.

### BRIEF DESCRIPTION OF THE FIGURES

FIG. 1 is a schematic representation of an example system.

FIG. 2 are sample schematics of the general shape of a rod-like orthopedic implant.

FIG. 3 is a picture of an example surgical nail implementation.

FIG. 4 is a schematic of an example head region of a surgical nail.

FIG. 5 is a schematic example of an end cap connection to the surgical nail head region.

FIG. 6 is a schematic of a solid surgical nail.

FIG. 7 is a schematic of a tubular surgical nail.

FIG. 8 is a schematic of an open surgical nail.

FIG. 9 is a schematic of a partially tubular, partially solid, and partially open surgical nail.

FIG. 10 is a schematic of an open segment of a surgical nail with electrode stimulation sites on the exterior of the surgical nail.

FIG. 11 is a schematic of an open segment of a surgical nail with an electrode stimulation site proximal to the opening of the surgical nail.

FIG. 12 is a schematic of an open segment of a surgical nail with an electrode stimulation site along the interior of the surgical nail.

FIG. 13 is an example schematic of the end cap connecting to the surgical nail body.

FIG. 14 is an example schematic of color-based positioning of electrodes.

FIG. 15 is an example schematic of electrode positioning using an insulation layer.

FIGS. 16-2o are example schematics of electrode positioning on a solid segment of a surgical nail.

FIG. 21-25 are example schematics of electrode positioning on an open segment of a surgical nail.

FIG. 26-28 are example schematics of electrode positioning on a tubular segment of a surgical nail.

FIG. 29 is a schematic of an example PCB that contains the implant circuitry.

FIG. 30 is an example schematic of a folded PCB.

FIG. 31 is an example schematic of a folded PCB implant circuitry within the end cap.

FIG. 32 is an exemplary system architecture that may be used in implementing the system and/or method.

### DESCRIPTION OF THE EMBODIMENTS

The following description of the embodiments of the invention is not intended to limit the invention to these embodiments but rather to enable a person skilled in the art to make and use this invention.

### 1. Overview

A system for an electrically stimulating orthopedic implant includes: a rod-like orthopedic implant; a set of electrodes positioned along the body of the orthopedic implant, wherein the electrodes are controllably connected to control circuitry; and an implant end cap, that is hermetically sealed and contains the control circuitry used in controlling the electrodes. The system function to provide a dynamic orthopedic implant that can provide electrical stimulation to stimulate tissue growth and analyze tissue.

The system may have a particularly useful implementation as a surgical nail orthopedic implant (also referred to as intermedullary rod or intermedullary nail), wherein the electrodes are exposed along the shaft of the nail, and the control circuitry is contained in the end cap at the head of the surgical nail. The system may thus enable dynamic targeted stimulation to subregions along the length of the nail and/or in different subregions around the nail. The set of electrodes preferably include distinct subsets of electrodes that are individually controllable. Individually controlled and isolated stimulation may be applied through these electrodes by controlling current magnitude and polarity. Additionally or alternatively, the surgical nail may provide sensing capabilities. The system enables impedance measurements through tissue in proximity to electrode sites along the nail. Through these impedance measurements, tissue structure and quality (e.g., identification of bone tissue and bone growth) may also be measured and monitored. The impedance measurements, in some variations, may be used to dynamically adjust stimulation.

The system may be particularly applicable to the field of orthopedic treatment, particularly in the field of surgical orthopedic treatment. The system may enable an enhanced surgical nail compatible with surgical practices. For example, the enhanced surgical nail of the system may enable, during surgical insertion, the use of a guide wire threaded through the inner chamber of the surgical nail. After positioning in the body, an end cap with the control circuitry can be inserted thereby enabling the stimulation/sensing capabilities of the system. The surgical nail system may be used in a variety of types of surgical orthopedic devices. Examples include: nails, rods etc. In this manner, this document will be primarily directed towards surgical nails, but may be applied to a more general field of orthopedic implants.

The system may provide a number of potential benefits. The system is not limited to always providing such benefits, and they are presented only as exemplary representations for how the system may be put to use. The list of benefits is not intended to be exhaustive and other benefits may additionally or alternatively exist.

The system may provide an “enhanced” implant that may potentially provide a better treatment as compared to a similar non-stimulating implant. By providing electrical treatment, an orthopedic implant potentially provides an improved treatment. This improved treatment may be implementation dependent. Examples of improved treatment may include: modifying tissue growth (e.g., increasing/decreasing bone growth) and adjusting biological behavior (increasing/decreasing heart rate, increasing/decreasing blood flow, increasing/decreasing gastrointestinal motility).

Additionally, the system may provide an improved method for tissue monitoring. Through use of the system, bone growth, and other tissue growth, may be more precisely monitored without the incorporation of the use of additional invasive procedures beyond inserting the implant. For example, impedance measuring capabilities enabled through dynamic control of the electrodes may enable monitoring of bone growth and/or tissue density in surrounding proximity of a surgical nail.

The system potentially provides the benefit of a seamless integration of electronics into a surgical nail, which is the load bearing device. Through the system, these electronic components may be shielded to provide the benefit of monitoring and treatment while simultaneously shielding a patient from their potentially toxic nature. In some variations, the dynamic circuitry components can be hermetically sealed within a biocompatible container (e.g., a titanium body of an end cap) and any exposed electronics would be conductive elements made from substantially biocompatible materials.

In some variations, modular electronic components may be incorporated with the surgical nail, which can then be removed later. This potentially provides the added benefit of an active treatment implant, while treatment is necessary while allowing the benefit of continued use of the surgical nail implant on a longer term that still provides a mechanical functionality.

The system may also enable the incorporation of other sensors within and along the orthopedic implant (e.g., temperature and stress sensors). These sensors may further improve the effectiveness of the surgical treatment by enabling more precise observation and response within the implant that does not require invasive measurement techniques.

The system potentially provides the benefit of customized treatment. The system may be customized in operation to deliver stimulation in selected regions. For example, the nail may include color-coded, or otherwise labeled regions along the length of the nail such that an operator can specify where and how stimulation should be applied to different sub-regions. In this manner, a doctor may set a surgical nail to deliver stimulation that is based on the patient's condition and on the manner of the patient's recovery.

### 2. System

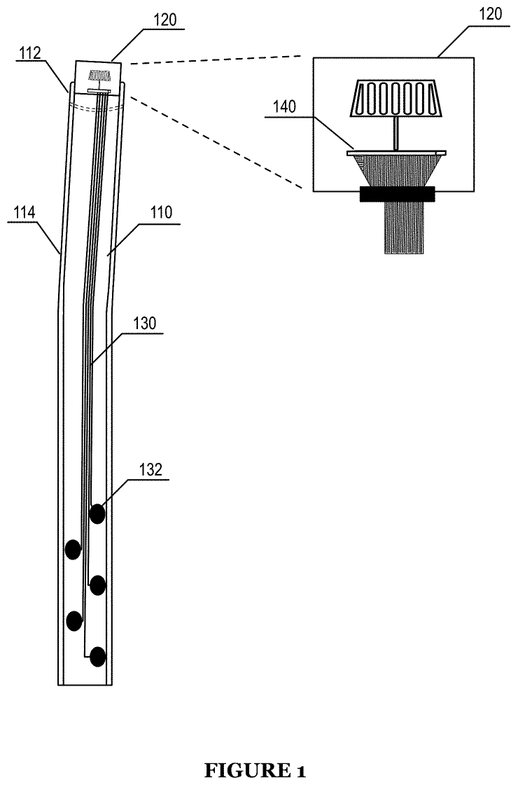

As shown in FIG. 1, a system for an electrically stimulating orthopedic implant includes: a rod-like orthopedic implant 110, comprising a shaft (also referred to as implant body) of the orthopedic implant, with distinct electrode sites situated along the implant body; and an end cap 120, comprising the head of the orthopedic implant, situated at one end of the shaft; a set of electrodes 130, individually controllable, wherein each electrode: includes a distinct stimulation site 132, comprising an active exposed segment of the electrode situated on an electrode site, is conductively coupled to an implant control circuitry, and is conductively isolated from all other electrodes in the set of electrodes; and implant circuitry 140, situated at least partially within the end cap, comprising: implant receiver circuitry, effective to convert an electromagnetic field to an electric current and implant control circuitry, configured to control current flow through the set of electrodes. The system functions as an orthopedic implant that is enabled to provide localized (e.g., sub-millimeter, millimeter, centimeter) and/or controlled electrical stimulation to the tissue around the implant. As an orthopedic implant that provides electric stimulation, the system may simultaneously provide multiple stimulations of distinctly controlled magnitudes along different regions of the implant. Furthermore, the directionality or polarity of stimulation may, in some variations, be controlled and varied.

Dependent on implementation, the system may have additional components which may alter or improve functionality. Examples of additional components include: screws (e.g., to connect/fasten the implant in place), power source(s), and sensors (e.g., pressure/stress sensors, temperature sensors).

The system may include a rod-like orthopedic implant no (also referred to as the implant body). The rod-like orthopedic implant no functions both in the role of the intended implant, in addition to functioning as an implant body that provides electrical stimulation. The rod-like orthopedic implant no may comprise any relatively “rod-like” shape and/or volume (i.e., shaft). The two ends of the shaft are the head-side and the tail; wherein the head-side of the rod-like orthopedic implant 110 is defined as the side that connects to the end cap 120, whereas the tail is defined as the end not connected to the end cap.

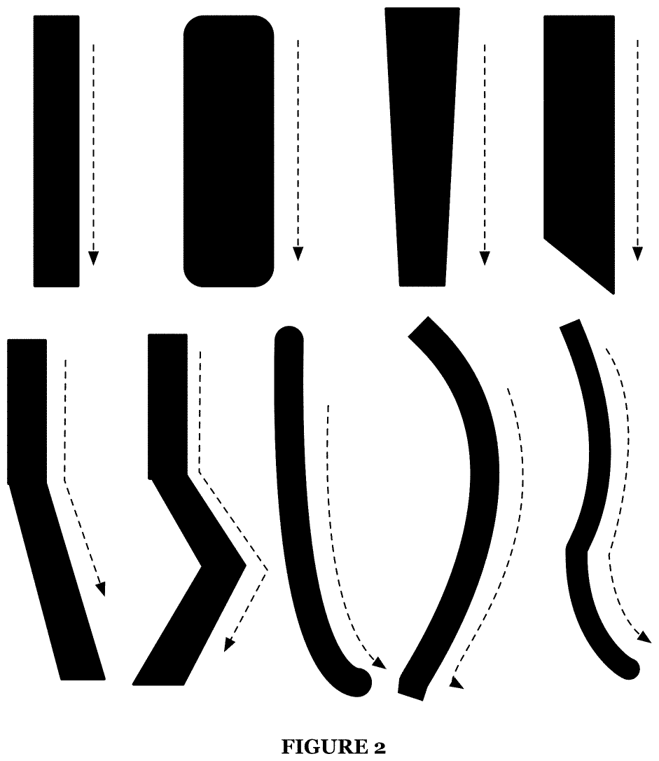

As used herein, rod-like orthopedic implant 110 may refer to any orthopedic implant that has a single predominant axis. That is, the implant body extends primarily along one axis (referred to as the length or the length axis), whereas the other dimensions of the rod-like orthopedic implant no may be several times shorter than the length. As used herein, the implant body spans a length at least 2 times as long, along the length axis, as compared to the other axes. Commonly, the implant body is at least 4-5 times longer along the length axis as compared to the other axes. The rod-like orthopedic may be completely straight, may be tapered along the shaft, may have one or more kinks (sharp bends along the length of less than 90 degrees), or may be curved in one or more directions. FIG. 2 shows nine crude two-dimensional projections layouts of possible rod-like orthopedic implants no shapes. Example shapes of the rod-like orthopedic implant 110 include: nails, rods, bars, etc.

As used herein, the length axis (or just length) of the rod-like orthopedic implant 110, follows the general length of the rod-like orthopedic implant, as shown with the dash lines in FIG. 2. That is, the length axis (or length) of the rod-like orthopedic implant 110 extends along the shaft, from the head to the tail of the rod-like orthopedic implant. The length may thus have kinks or curves as per the rod-like orthopedic implant 110.

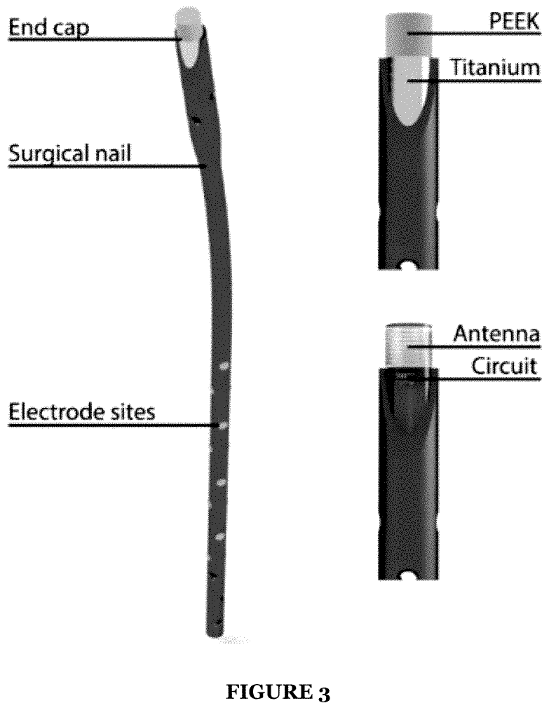

In many variations, the rod-like orthopedic implant 110 comprises a surgical nail. Without loss of generality, for simplicity, the rod-like orthopedic implant 110 will generally be described as a surgical nail, but the system may be implemented as any broad class of rod-like orthopedic implant no. The surgical nail (also referred to as intermedullary rod or intermedullary nail), may be of any typical, or non-typical shape or size dependent on the required implementation. For example, the surgical nail may be straight, bent, solid, hollow, include openings, etc. The surgical nail comprises the shaft of the orthopedic implant, wherein one end connects to the end cap (i.e., the head) and the opposite end comprises the tail end and the elongated portion extending from the head to the tail is described as the “length” of the nail. As a surgical nail, the tail end may have an equal or lesser cross-sectional area as compared to the head end. One example illustration of the surgical nail is shown in FIG. 3. The surgical nail may have any common, or uncommon, attachments, such as screws and fasteners.

The surgical nail is preferably constructed of a durable non-toxic, minimally corrosive, material. In some variations, the surgical nail is composed of titanium. Additionally or alternatively, the surgical nail may include other non-toxic metals, or metal alloys, such as: titanium alloy, platinum, stainless steel, cobalt-chromium alloys, tantalum, and/or any combination of thereof. The surgical nail may additionally or alternatively, be at least partially composed of non-metallic compounds, such as: biomedical tissue, silicone, or plastics (e.g., polyether ether ketone (PEEK)).

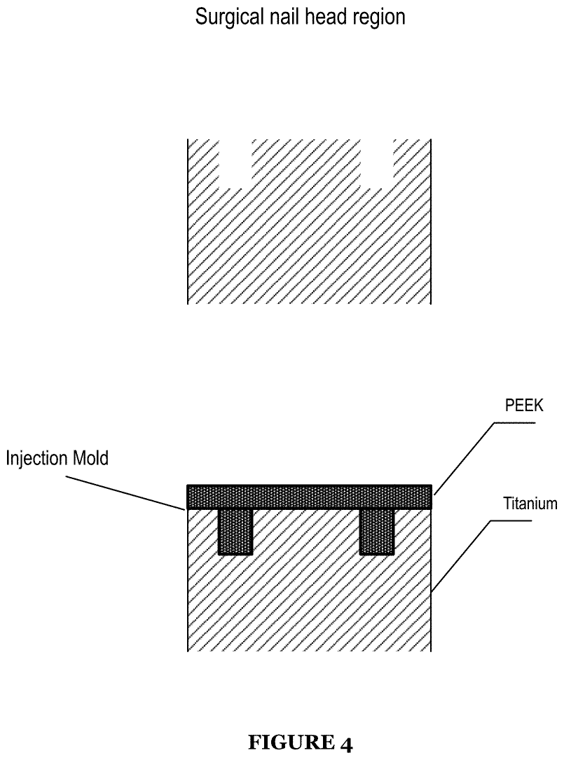

The head (or head region) of the surgical nail may comprise an opening cavity that enables attachment of the end cap 120. Alternatively, the head region may not include an opening cavity. The head region may be composed of the same material as the rest of the surgical nail (e.g., titanium), or may be composed of different materials (e.g., PEEK) or combinations of different materials. For example, as shown in FIG. 4. the head region may be partially composed of titanium and PEEK, wherein the PEEK region includes extensions into the titanium region, thereby fastening the PEEK into place (e.g., through injection molding). Alternatively, the titanium region may extend into the PEEK region. In some variations, the head region cavity may be threaded to enable the end cap 120 to be “screwed on”. Other attachment mechanisms (e.g., mechanical fastener) may alternatively be used to enable an end cap 120 to be attached to the head region to mechanically and conductively couple the end cap to the surgical nail. Additionally or alternatively, the head region may be lined by some material to provide a seal with the end cap (e.g., PTFE tape, or a washer). Additionally or alternatively, the head region may be composed of a material that enables the use of an adhesive to fix the end cap 120 in place.

In some variations, the head region may include a conductive connector. The conductive connector functions as the electrical and mechanical connector between end cap 120 and the rest of the surgical nail. In this manner, the conductive connector preferably includes electrical conduits that connect to the circuitry within the end cap and to the set of electrodes within the surgical nail. Additionally or alternatively, the conductive connector may include locking mechanism(s) to enable end cap and/or surgical nail attachment. The conductive connector may be produced in a similar fashion as the end cap and may be composed of plastic and/or metal. In some variations, the conductive connector is shaped such that once attached to the end cap 120, the end cap is sealed, preventing fluid ingress. In some variations, the conductive connector is shaped such that once attached to the end cap and the surgical nail, the electrical circuitry conductively connects the end cap circuitry with the surgical nail circuitry. In some implementations, the conductive connector furthermore forms a hermetic seal with the end cap.

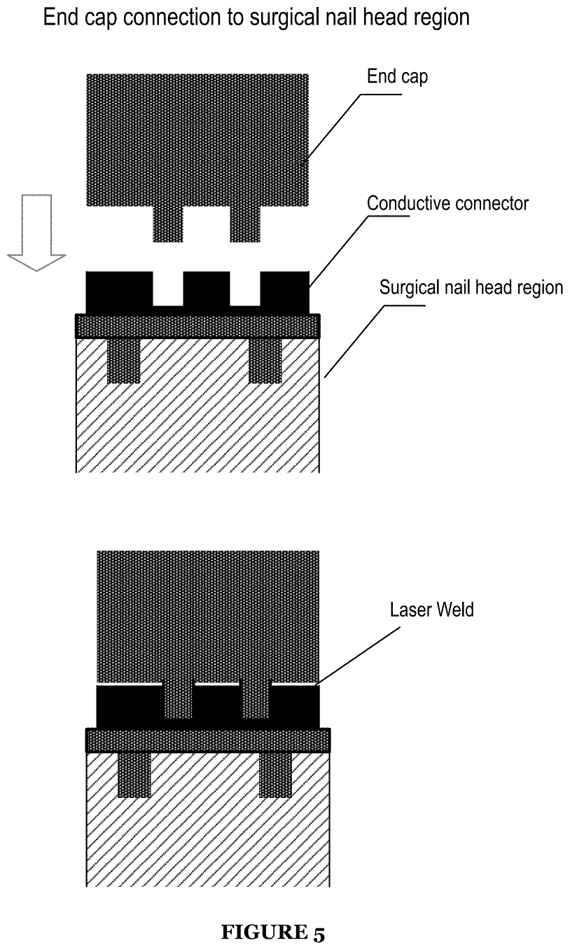

The conductive connector may comprise any kind of material (for example a biocompatible metal such as titanium or platinum) that can electrically connect to the implant circuitry 140 within the end cap 120 such that once the end cap is fastened to the head, current may travel between the implant circuitry and the connectors in the head region of the surgical nail. Dependent on the implementation, the conductive connector may include direct connectors (e.g., wiring, circuit board, conductive plates), or indirect connectors (e.g., inductive coupling, capacitive coupling). One example of how the conductive connector is situated and connected to the end cap 120, is shown in FIG. 5, wherein in this example the connective connector is embedded in PEEK material and injection molded to the head region of the surgical nail. In some variations, the metal constituting the conductive connector is made of a different material than the electrode sites. In some variations, the direct current voltage required to drive substantial current (over 30% of total stimulus current) through the conductive connector is higher than that required to drive substantial current through the electrodes, if both are exposed to fluid. In these variations, substantial current may not be sourced or sinked by the conductive connector in such cases that it is exposed to bodily fluid. This may, for example, be the case in example implementations where the connector is made from titanium and the electrode sites are made from platinum.

The connector piece functions as the electrical and mechanical connector between end cap and the rest of the surgical nail. In this manner, the connector piece preferably includes electrical conduits that connect to the circuitry within the end cap and to the set of electrodes within the surgical nail. Additionally or alternatively, the connector piece may include locking mechanism(s) to enable end cap and/or surgical nail attachment. The connector piece may be produced in a similar fashion as the end cap and may be composed of plastic and/or metal. In some variations, the connector piece is shaped such that once attached to the end cap, the end cap is sealed from fluid ingress. In some variations, the connector piece is shaped such that once attached to the end cap and the surgical nail, the electrical circuitry conductively connects the end cap circuitry with the surgical nail circuitry. In some implementations, the connector piece furthermore forms a hermetic seal with the end cap.

The surgical nail body, i.e., the shaft region, may be an elongated shape. The surgical nail body serves as the primary volume of the orthopedic implant. The surgical nail body may be straight, curved or elongated along any suitable path. In some variations, the surgical nail body may include holes approximately orthogonal to the length of the surgical nail. These holes may function to enable screws or other components to fix the surgical nail in place.

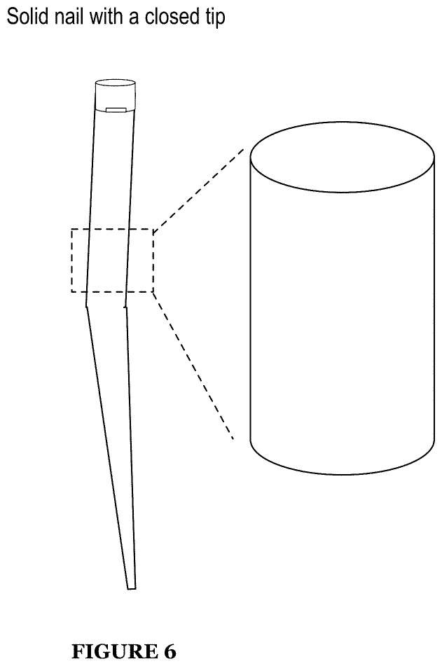

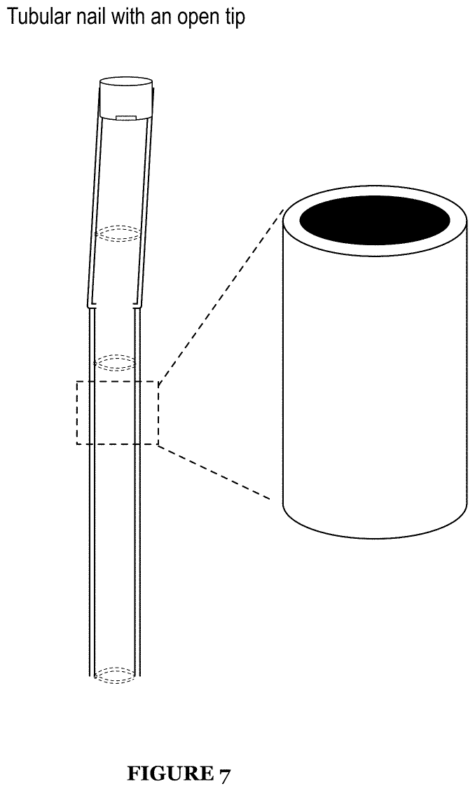

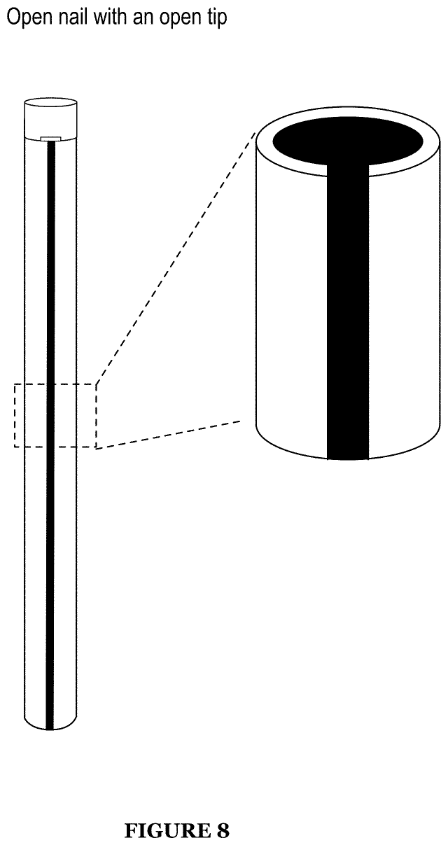

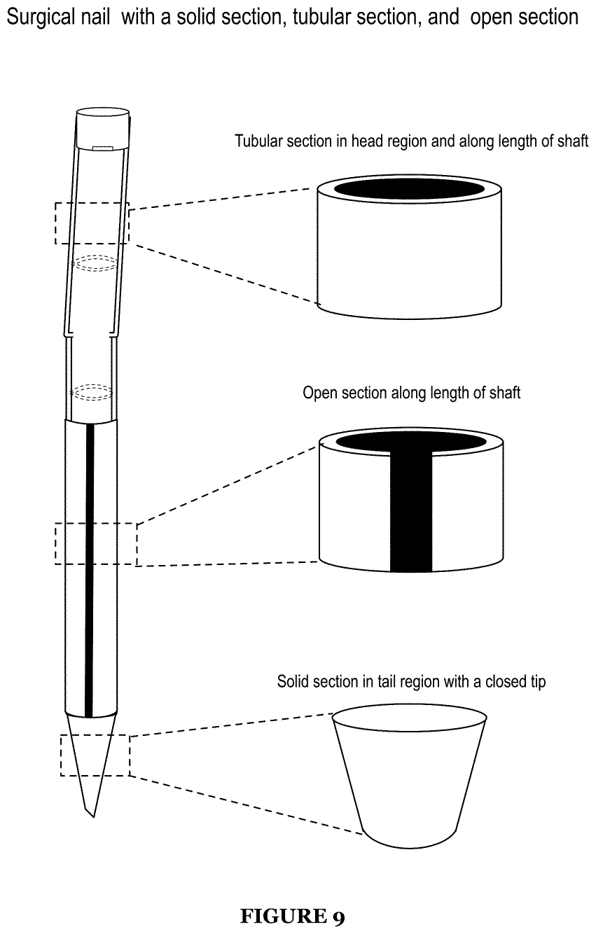

Dependent on implementation, the surgical nail body may be solid, as shown in example FIG. 6; tubular (i.e., include an internal cavity), as shown in example FIG. 7; be open, as shown in example FIG. 8; and/or some combination of the three, as shown in example FIG. 9. That is, dependent on implementation the shaft of the surgical nail may be: entirely solid, tubular, or open; or comprise any combination of solid, tubular, and/or open sections. Independent of the shaft composition, the tail may be open or closed.

The shaft of a tubular nail or a tubular section may be substantially hollow, wherein the shaft includes a defined internal cavity extending along the tubular section of the nail. In many variations, the proximal end, i.e., head, comprises an opening of the internal cavity. Dependent on implementation, the internal cavity may also have an opening at the distal end. This opening may be as large, or smaller than the opening at the head. The size (i.e., cavity diameter, or cross-sectional area) of the cavity may vary dependent on implementation. In some variations, the cavity may comprise a significant cross-sectional area of the surgical nail. In other variations the cavity may be significantly smaller (e.g., just sufficient to enable wire to pass through the interior of the surgical nail.

The shaft of an open nail or an open section of a nail may also be substantially hollow, wherein the shaft includes a defined internal cavity. Additionally, open segments include an “opening” such that along the open segment of the nail, the interior surface and the exterior surface form a continuous surface. In the example FIGS. 8 and 9, the opening is shown as a slit along the length of the nail, but generally, the opening may have any desired shape dependent on implementation. For example, in variations, where it is desired that biological mass eventually envelops the surgical nail, the open section may span the entire nail to enable biological material to completely grow into the nail. In another example, the open sections may comprise small holes enabling electrodes within the nail to be exposed on the exterior of the surgical nail.

The orthopedic implant 110 may have electrode sites. Electrode sites may comprise regions of the orthopedic implant 110 that can be fitted with electrodes. The electrode sites may vary dependent on implementation. For surgical nail variations, the electrode sites may be primarily situated on the shaft of the nail. Additionally or alternatively, the head and the tail may also include electrode sites. The shape and positioning of the electrode sites may vary dependent on implementation. For example, on solid regions of the surgical nail, electrode sites may comprise “etchings” on the exterior surface of the shaft, such that an electrode 130 may be fitted into (or onto) the etched region(s). In tubular sections of the shaft, electrode sites may be fitted in holes (or other openings) along the body of the shaft such that electrode “wiring” may travel through the tubular region with the electrodes situated exposed on the exterior surface of the shaft. Holes, or open sections, may also be used to aid in connecting wiring traveling though the tubular region to electrodes sites exposed on the exterior surface of the shaft. For open-sections of the of the shaft, electrode sites may be situated anywhere within (along the open-section), or along the shaft.

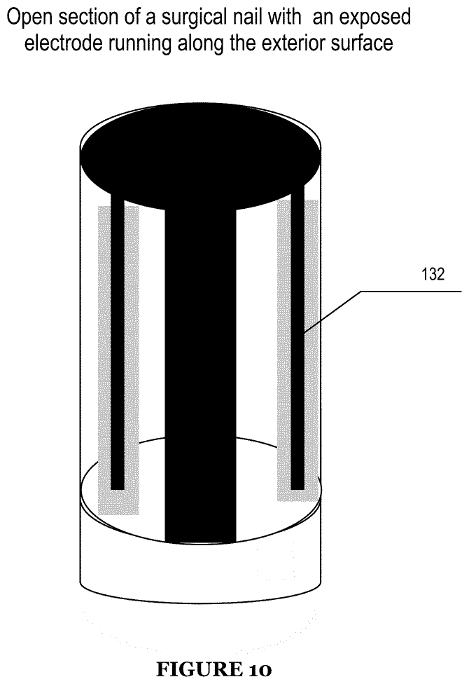

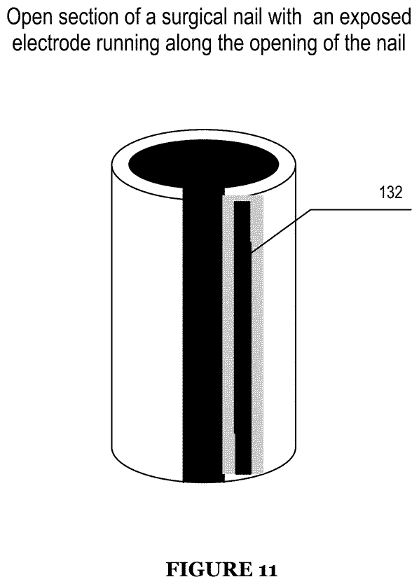

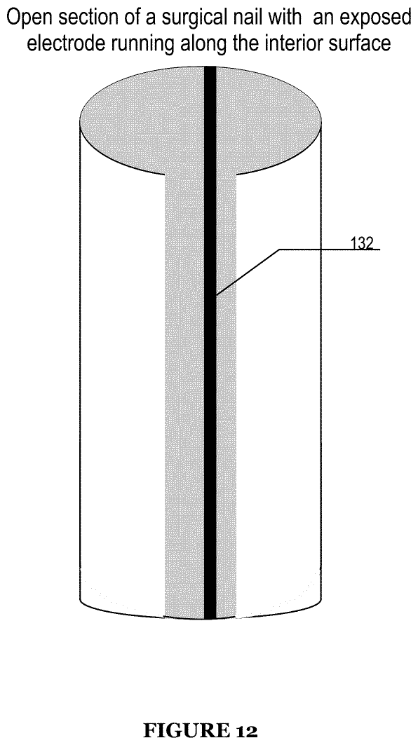

For example, in one open-section implementation, one electrode site may comprise the entire length of the open section. In different implementations, electrode sites may be along the exterior surface of the open section, along the opening of the open section, on the interior of the open section but sufficiently exposed to the exterior, or any combination thereof. In one example, as shown in FIG. 10, the electrode site extends along the exterior surface of the surgical nail. In another example, as shown in FIG. 11, the electrode site extends along the opening of the surgical nail. In another example, as shown in FIG. 12, the electrode site extends along the interior surface of the open section. Although shown directly opposite the opening, the electrode site may be anywhere along the interior surface and may be positioned such that it is sufficiently close to the opening of the open section to affect an electric field on the exterior of the surgical nail. Dependent on implementation, exposed electrode sites may be situated on, or have an insulation covering or layer to separate conductive components (e.g., separating the orthopedic implant 110, and each electrode from the set of electrodes 130. This insulation covering (discussed further below) may be implementation specific and dependent on the implant composition, electrode positioning, and desired type(s) of stimulation.

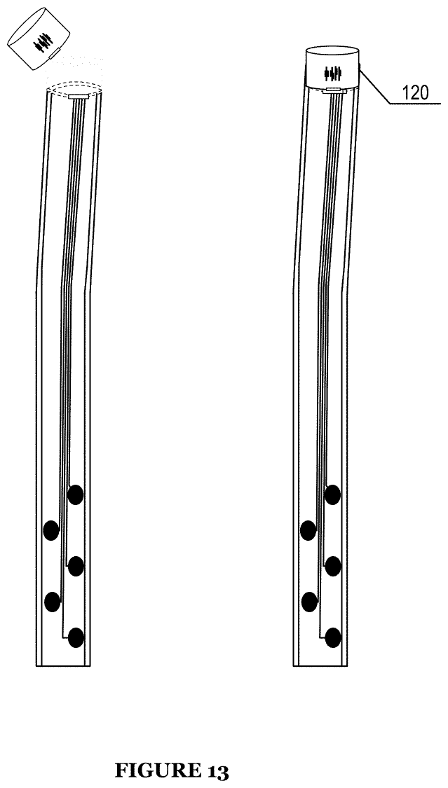

The system may include an end cap 120. The end cap 120 functions as a housing that contains the implant circuitry 140 and electronic components of the system. The end cap 120 may be directly connected to the rod-like orthopedic implant no. In surgical nail variations, the end cap 120 may directly connect and/or fastened to the head region of the surgical nail, as shown in FIG. 13. Dependent on implementation, the end cap 120 may be permanently fixed in place or further detachable from the implant. As a housing for electronic components, the end cap 120 may be sealed, such that biological material does not flow into the end cap 120 and any type of electronic residue (e.g., battery solution) does not leak out of the end cap. In some variations, the end cap 120 is hermetically sealed.

The end cap 120 may include connectors such that electric components sealed within the end cap 120 are electrically connected to electrodes, and other components, outside of the end cap. In some variations, this connection may comprise an electric connection to the complimentary connectors within the head region of the surgical nail.

In surgical nail variations, the end cap 120 may fasten to the head of the surgical nail. Dependent on the implementation, this may include “screwing” the end cap 120 (e.g., wherein the end cap has threaded region that may be screwed into the head region). Additionally or alternatively, the end cap may be mechanically fit into the head region. For example, the end cap 120 (or the head region) may have a snap fit such that the end cap is securely fixed into the head region. Additionally or alternatively, the end cap 120 may be attached to the head region in some other way. Examples include: attached using an adhesive, molded into place within the head, attached using a locking mechanism, etc. In some variations, as shown in FIG. 5, the end cap 120 may attached to a conductive connector (e.g., by welding) of the rod-like orthopedic implant 110, such that the components in the end cap are hermetically sealed while still maintaining an electrical connection with the rest of the implant body.

The end cap 120 may be composed of any non-toxic material. In some variations, the end cap 120 may include conductive regions (e.g., electrode sites) similar to electrode sites situated on the shaft on the surgical nail. In some variations the end cap 120 is composed of PEEK. In PEEK variations, the end cap 120 may be directly overmolded around the internal components. In some of these variations, the end cap 120 may be first constructed as two (or more) pieces. The internal components may then be situated onto these end cap 120 pieces prior to combining the pieces (e.g., by adhesive, welding, or overmolding) to form the completed end cap.

In removable variations of the end cap 120, the conductive regions (e.g., electrical conduits to conductively couple to the electrodes on the shaft of the surgical nail) can be sealed, which functions to prevent or reduce the effects of fluid infiltration. Sealing or other approaches of conductive insulation can prevent the conduits from acting as electrodes and to prevent current from unintentionally crossing between different conduits through the fluid. In one alternative, the end cap 120 may include wireless conductive components so that the end cap can induce current in the electrodes of the surgical nail without direct conductive contact.

In some variations, the end cap 120 may include a secondary housing module. The secondary housing module may function to provide a distinct circuitry housing. In some variations, the end cap 120 may include multiple secondary housing modules. This distinct housing may be used to separate electrical components such that they do not interfere with each other, and/or to provide a “better” position for positionally dependent electronic components (e.g., an antenna). Dependent on the implementation, the secondary housing module may be directly connected to the primary housing module (i.e., the end cap 120 as described previously), or the secondary module may be situated on the implant distinctly to the primary housing module. For example, the secondary housing module may be situated within a tubular surgical nail, on the tip of a surgical nail, or any other region of the rod-like orthopedic implant 110 as desired.

In some variations, the secondary housing module may have a distinct composition. This distinct composition may help improve or add additional functionality For example, the first housing module may be composed of non-conductive material (e.g., PEEK), to reduce disruption of communication components (e.g., an antenna), whereas the secondary housing module may be composed of material to provide of strong support material (e.g., titanium) to provide better stability and protection for the internal circuitry (for example by benefiting from hermetic sealing implemented using titanium to titanium welding). In this variation, the end cap 120 may include a PEEK portion housing the antenna and a titanium housing the implant circuitry 140. An antenna may be conductively connected to the implant receiver circuitry through sealed connectors. The implant control circuitry may then be conductively connected to exposed connectors for conductive coupling to the electrodes when inserted into the head of the rod-like orthopedic implant 110.

The system may include a set of electrodes 130. The set of electrodes 130 function to provide electrical stimulation (e.g., for treatment). Each electrode, from the set of electrodes, includes a distinct stimulation site 132 and circuitry conductively coupled to the implant control circuitry within the end cap 120.

Each electrode, from the set of electrodes 130, may be individually controllable, such that any direction of current of a desired magnitude may be sent or received from each electrode. In this manner, a single electrode, multiple electrodes, or the entire set of electrodes 130 may function identically, individually, and/or in a complementary fashion (e.g., one subset of electrodes may be set to function as current sources, sending a current to another subset of electrodes set to function as current sinks.

The shape, size, and number of electrodes may be implementation specific. Variations may depend on the implemented rod-like orthopedic implant 110, and the desired treatment to be given. In some variations, as shown in FIG. 3, the stimulation sites may comprise round pads on the exterior of the shaft of the surgical nail. In one example of this variation, the surgical nail may have a set of eight electrodes, with round pad stimulation sites, positioned around the shaft 112. These pads may be composed of non-toxic conductive material (e.g., titanium). In some variations, the electrode sites on the shaft may be shaped such that the electrode stimulation sites lock into the electrode sites. Alternatively, electrode may be molded, adhered, soldered, or otherwise attached into each electrode site (potentially with some insulation between the electrode site and the rest of the shaft). In another variation, the stimulation sites may comprise flexible/semi-flexible metal plates that are folded through the electrode sites such that they stay fixed in place. In one variation, for an open surgical nail region, the electrode site may comprise conductive plates directly exposed from the interior of the surgical nail. In another variation, the stimulation sites may include conductive etchings on the surface of the surgical nail (e.g., a conductive etch on the surface of the solid surgical nail.

Common variations of electrode stimulation sites 132 include round pads and linear pads, but electrode stimulation sites may generally have any shape, preferably dependent on implementation. Dependent on implementation, the actual shape of the stimulation site may vary greatly. For example, electrode stimulation sites 132 may comprise metal pads (of any shape), wire rings around the circumference of the surgical nail, wire lines along the length of the shaft, and/or any complex shape of wiring in, or on, the surface of the surgical nail. In some variations, parts of the end cap 120 may also include stimulation sites (e.g., electrode sites may sit on the surface of the end cap 120, and/or extend from the end cap into nearby soft tissue).

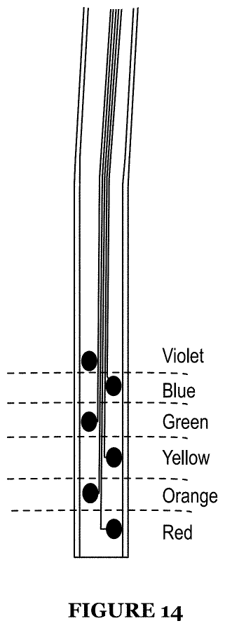

In some variations, each electrode, or subsets of electrodes, from the set of electrodes 130 may have specific regional designations. The regional designations may function to improve personal treatment (e.g., by a doctor), by quick identification of electrodes, or groups of electrodes. Regional designations may include giving the electrode, or groups of electrodes, color, graphical, or other imageable identifiers to indicate subsets of electrodes. The regional designations may be used so that a doctor, or another administrator, could more easily specify stimulation settings for subsets of the electrodes. The different regional designations can be used as specified input into operational controls of the device. For example, a doctor may indicate that three regional designations (e.g., three specific color bands of five color bands) should be activated for stimulation. The regional designation can serve as a more convenient approach and method for input. For example, this would be giving a color designation dependent on the positioning along the shaft. For example, electrode stimulation site 132 may be positioned along the length of the shaft of the surgical nail. In one implementation, as shown in FIG. 14, the electrode(s) closest to the tip would be designated red, and electrodes closest to the head designated violet, and electrodes in between would be designated by colors of the spectrum in between. That is, a color coincides to a lengthwise position along the shaft of the surgical nail. In another example, color specification may be dependent on the region in the body (or bone). For example, electrodes facing into the bone would have one color designation (e.g., red), electrodes facing outward from the bone would have another color designation (e.g., blue), and all other electrodes would have an independent color designation (e.g., yellow). Other types of designations instead of colors may be additionally or alternatively implemented.

In one variation, an alternative type of regional designation can be configured for regional marking when imaged using a medical imaging technique. A medically imageable designation may use paint, coating, physical markings (e.g., surface patterning, physical surface forms, and the like), material patterning, and/or other techniques such that different subregions appear as distinguishable subregions in the imaged output when a medical imaging method is used like ultrasound, MRI, x-ray, or other suitable medical imaging technique. In this approach, a medical image may be generated of a patient and then the resulting image will have distinguishable regions (which can be visually distinguished by a doctor or other care giver in the imaging output). These imageable designations may then be used as control input to specify stimulation modes for different subregions of electrodes.

The set of electrodes 130 may include electrode circuitry. Electrode circuitry functions as the electrical conduit between the electrode stimulation site 132 of each electrode to the implant control circuitry within the end cap 120. The electrode circuitry can be one or more conductive traces, wires, and/or other suitable conductive paths connecting an electrode stimulation site 132 to the implant control circuitry. Additionally or alternatively, the electrode circuitry may connect to other components within the rod-like orthopedic implant 110. That is, the electrode circuitry electrically connects the end cap 120 components to the set of electrodes 130. Dependent on implementation, the electrode circuitry may comprise physical wiring, conductive traces on a flexible or rigid circuit board, conductive paths manufactured into the surgical nail body, silicon wafers, and/or any other type of durable electrically conducting material. In variations, where the surgical nail includes tubular and/or open regions, the electrode circuitry may travel through the interior of the surgical nail. The electrode circuitry may travel straight through the implant body, or it may be threaded through the shaft. Additionally or alternatively, the electrode circuitry may be at least partially etched or embedded on the surface of the surgical nail. In another variation, the electrode circuitry may comprise conductive traces on the internal surface of the tubular (or open-section) of the shaft.

The system may further include insulation. Insulation functions to electrically separate each electrode and other conducting components. As electrodes may be activated independently, insulation may be significant in preventing or directing current flows in proximity of the rod-like orthopedic implant 110. For a titanium orthopedic implant no, or other conductive implant body compositions, the insulation may electrically separate the set of electrodes 130 from the implant body. The inclusion of insulation may be particularly important around electrode stimulation sites 132. As the region where electric current is released, insulation may play an important role of electrically isolating each electrode stimulation site 132 from the electrode sites on the implant body. In one variation, insulation is situated on each electrode site thereby conductively isolating the electrode stimulation site from the surgical nail. Insulation can additionally be used to insulate the electrode circuitry (e.g., the conductive paths) from the surgical nail body and/or the body of the subject.

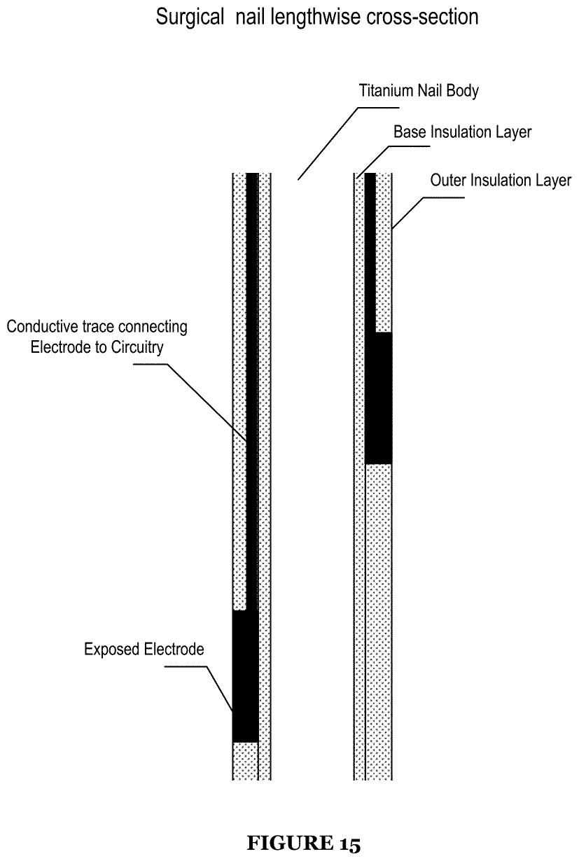

This insulation may comprise a coating, on the implant (or electrode), or may include an additional material layer. In one example of a material layer implementation, as shown in FIG. 15, the rod-like orthopedic implant no (e.g., solid, tubular, and/or open) may have material layer insulation coating (i.e., base insulation layer) on the orthopedic implant outer surface. The set of electrodes 130 may include electrode circuitry and stimulation sites positioned onto this base insulation layer (e.g., positioned by etching, printing, sputtering, etc.). These set of electrodes 130 may then be covered with an additional material layer insulation coating (i.e., outer insulation layer), wherein only the stimulation sites of the electrodes would be left exposed on the exterior of the implant. The insulation may be composed of any non-toxic, non-conductive material. In some variations, multiple forms of insulation are used. Examples include surface coatings (e.g., polyimide, epoxy, silicone, PEEK) and wire sheaths.

The insulation may be applied to the system during or after assembly of the entire system. For electrodes, insulation may be pre-acquired for circuitry (e.g., obtaining insulated wires), or may shaped around the electrode during electrode deposition into/onto the implant. For example, to prevent electric conduction between the electrode(s) and the implant body, polyimide insulation may be applied to the surgical nail implant prior to positioning of the electrodes in place. In other variations, the insulation may be packed around the set of electrodes 130. For example, the set of electrodes 130 (or subsets of electrodes) may be packaged with insulation which is then inserted into the surgical nail.

In some variations, the electrode sites are electrically isolated. That is, dependent on the composition of the orthopedic implant 110, the electrode sites may be constructed, or coated, with a material such that electrodes positioned in, or on, the electrode sites are electrically isolated from the body of the rod-like orthopedic implant no. Thus, the insulation composition, and/or coating, may extend sufficiently beyond electrode sites, along the rod-like orthopedic implant no, to prevent current from flowing directly from an electrode to the implant body. In some variations, the insulation composition, and/or coating, may additionally prevent immediate current exchange between any number of adjacent electrodes from the set of electrodes 130. This may, for example, alleviate current shunting from one electrode to the next through the surgical nail implant body in situations where transient currents are passed between electrodes (e.g., during impedance measurements). Thus, such coatings may result in more accurate impedance measurements where less current is shunted through the implant body and more current through the tissue.

As described above, the set of electrodes 130 may be positioned as desired per implementation, wherein the electrode stimulation sites 132 (or exposed electrodes) may be of any desired shape or size. The only constraints on electrode positioning is that they are conductively isolated and conductively coupled to the implant control circuitry. Herein example implementations are shown for segments of solid shaft, open shaft, and tubular shaft, implant bodies.

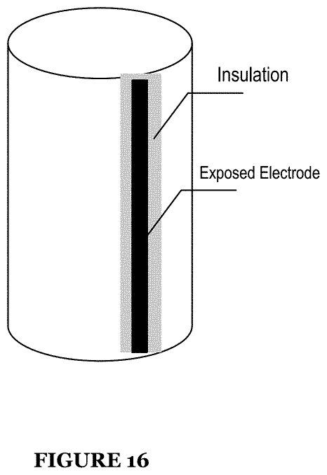

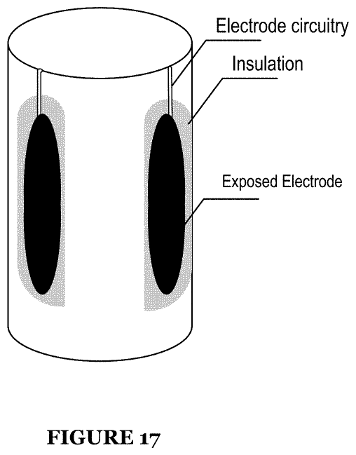

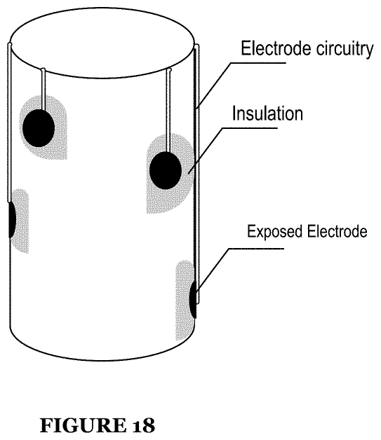

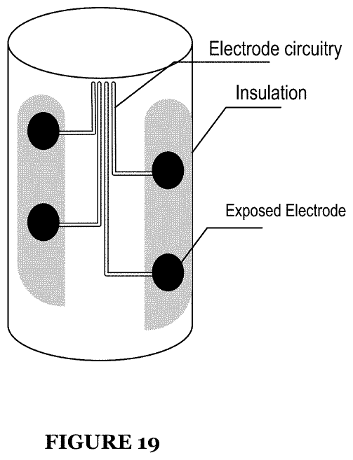

As shown in examples FIGS. 16-20, for a solid shaft segment, the electrode circuitry may be housed on the exterior of the rod-like implant body 110. The circuitry may comprise, etchings, traces into the implant body, and/or may comprise positioning of the electrode circuitry on an insulation layer, as shown in FIG. 15. Depending on implementation the circuitry may comprise “bundle” circuitry (all grouped together) or may comprise individual traces along the surface of the implant body. The electrode stimulation sites 132 may be positioned on the insulation covered electrode sites. Dependent on implementation, the circuitry may be required to pass through an electrode stimulation site 132, as shown in FIG. 20. For example, the electrode stimulation sites 132 may be positioned along the length of the surgical nail, wherein each electrode stimulation site may comprise a ring around the shaft of the surgical nail. In these variations, multiple layers of insulation may be incorporated to separate the exposed electrodes and the electrode circuitry.

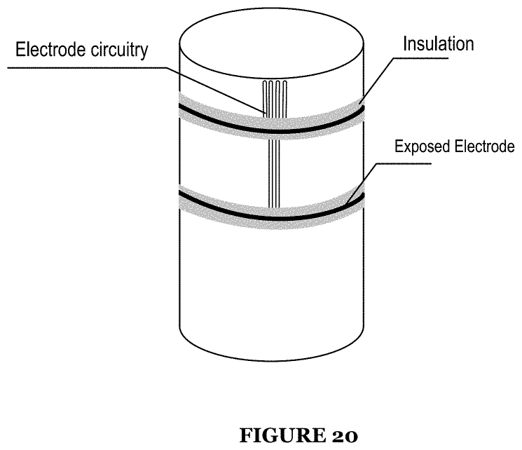

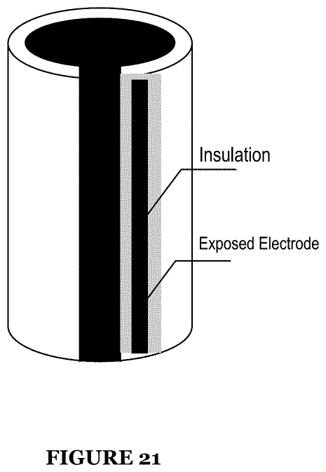

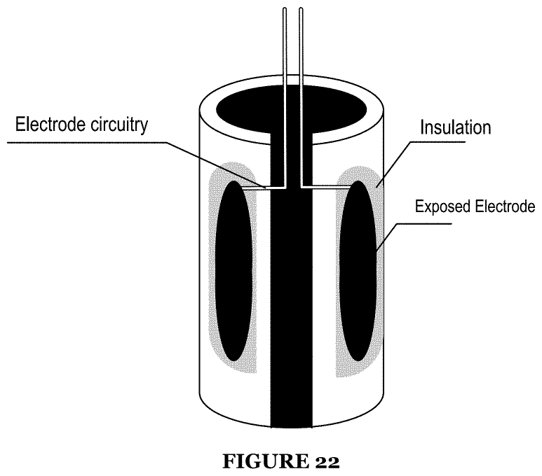

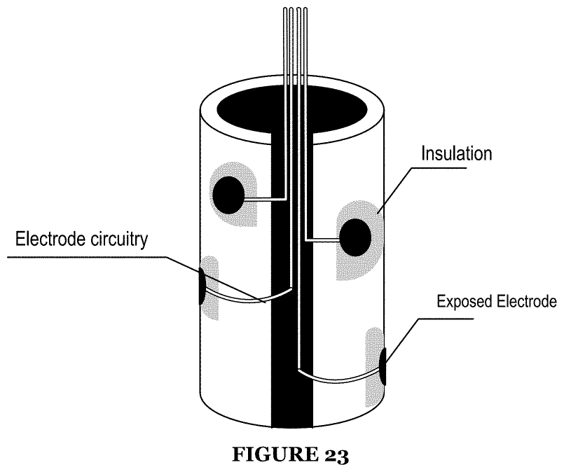

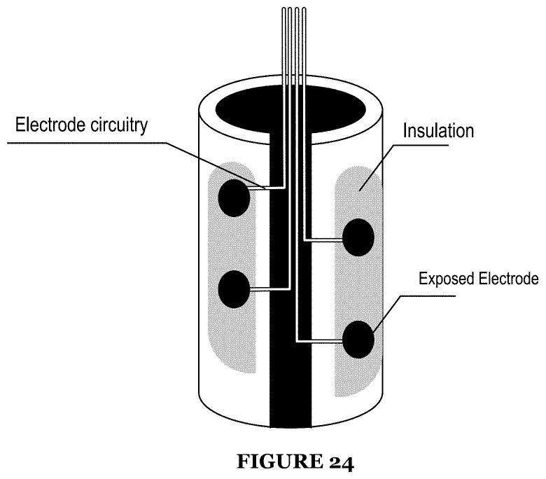

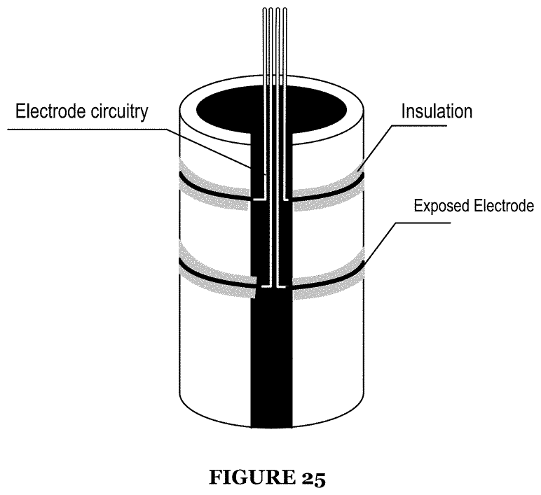

As shown in examples FIGS. 21-25, for an open segment, the electrode circuitry may be housed on the interior, exterior, or along the opening of the of the rod-like implant body 110. The circuitry may comprise bundles, or individual wires, in the interior of the implant body, but may also comprise etchings, traces on the exterior implant body, as per a solid segment. As per the circuitry, the electrode stimulation sites 132 may be positioned on the exterior, interior, and/or sufficiently proximal to the open region such that electrical stimulation may be implemented on the interior or exterior of the implant body.

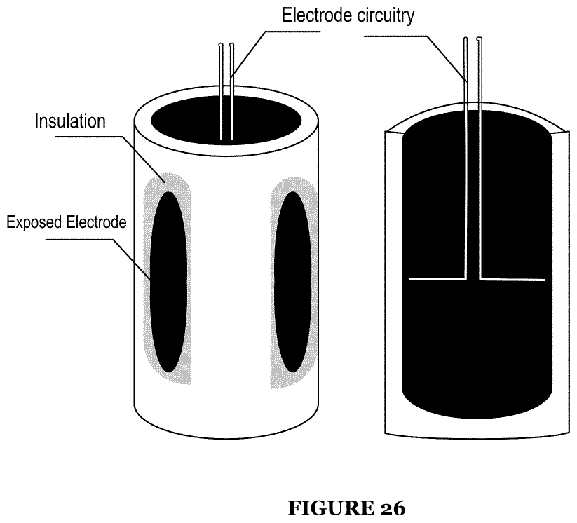

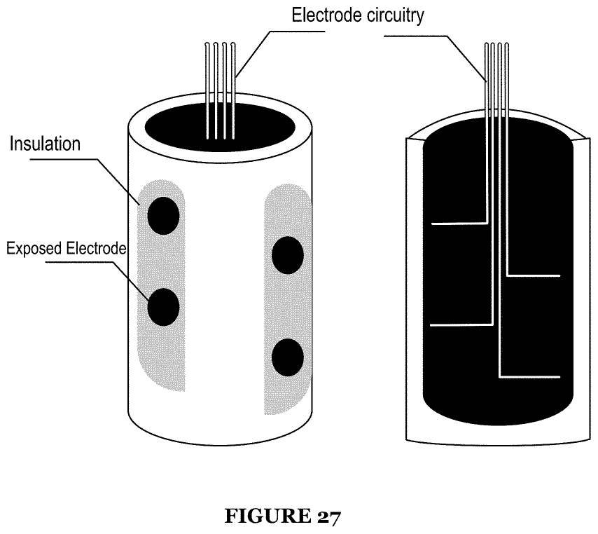

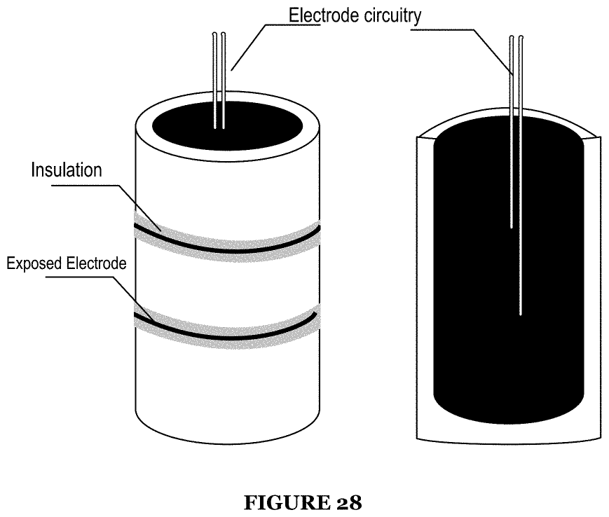

As shown in examples FIGS. 26-28, for a tubular segment, the electrode circuitry may be generally housed on the interior of the implant body, but may also be positioned on the outside as per the solid segment. The circuitry may comprise, bundles or individual wires in the interior of the implant body. The electrode circuitry connects to the electrode stimulation sites 132 via holes in the shaft beneath the electrode sites.

The system may include implant circuitry 140. The implant circuitry 140 is situated, at least partially, within the end cap 120. The implant circuitry 140 functions as the means of controlling operation of stimulation of the orthopedic implant. The implant circuitry 140 includes: implant receiver circuitry, effective to convert an electromagnetic field to an electric current; and implant control circuitry, configured to control current flow through the set of electrodes. Dependent on implementation, the implant circuitry 140 may include other components (e.g., sensors, batteries).

The implant circuitry 140 may be at least part embedded within the end cap 120. In many variations, the implant circuitry 140 is primarily situated on one, or more, printed circuit boards (PCBs) or some type of integrated circuit (IC). In one variation, the implant circuitry 140 is based on or connected to the PCB (e.g., the implant circuitry is situated on the PCB). In another variation, the implant circuitry may include an integrated chip, wherein electronic components are built onto, or connected to the IC. The PCBs function to provide a circuitry surface for the implant circuitry and enable easy connection between electronic components.

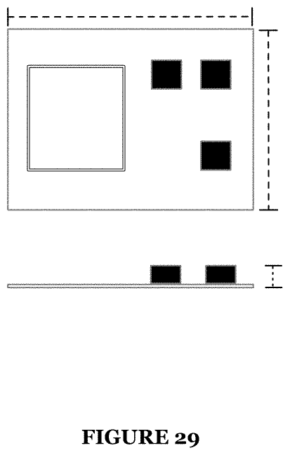

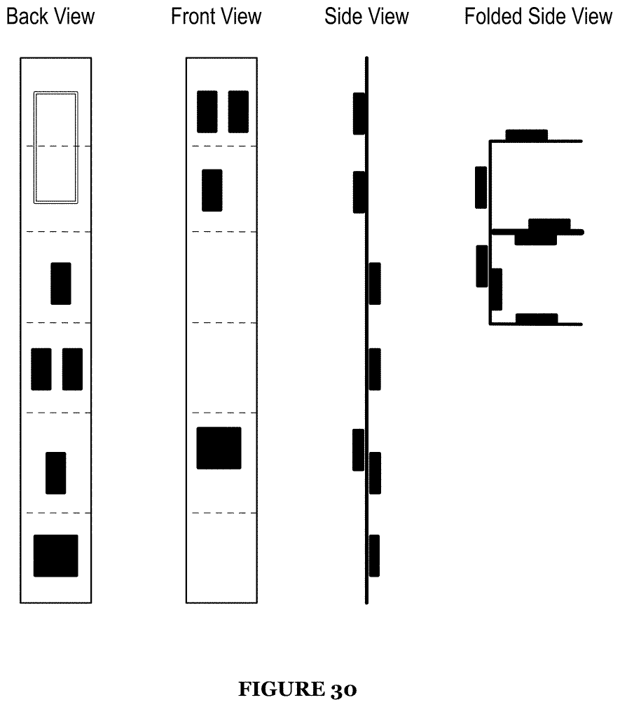

In some variations, the circuitry surface comprises at least one PCB as shown in FIG. 29; a schematic drawing of a single layer PCB from a front view (top) and side view (bottom). The PCB can be single-sided, double-sided, and/or multi-layered, wherein each side/layer may contain electronic components embedded in, or on, the surface of the PCB. In some implementations, the PCB is single sided and single layered. In other implementations, the PCB may be multi-sided as shown in FIG. 30; A schematic drawing of a multi-sided PCB from a front view, back view, side view, and folded view. In preferred variations, the PCB itself may be flexible, wherein all or parts of the PCB are bendable (although electronic components on the PCB may not be bendable). In other words, the PCB includes a flexible substrate. As the PCB is preferably part of a medical implant, the PCB may be constructed of any appropriate non-toxic non-reactive material. In preferable variations the PCB is constructed of polyimide, although other non-toxic, non-reactive materials may be alternatively used.

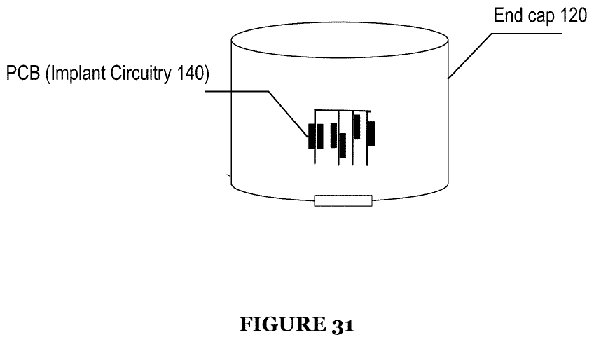

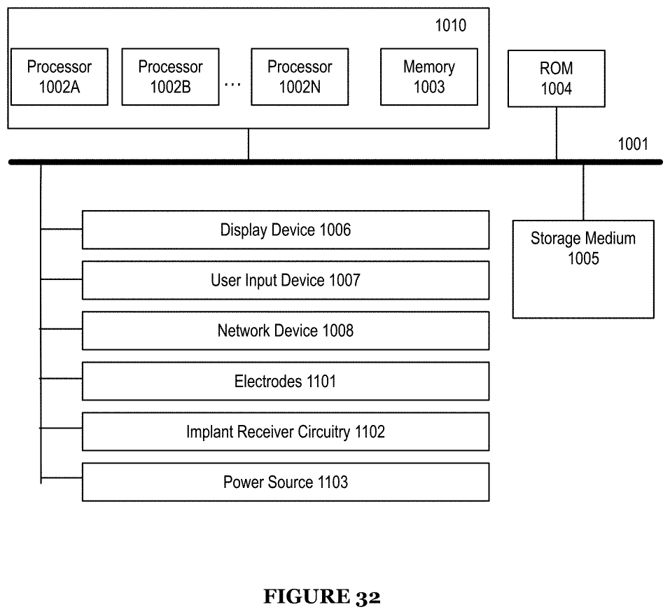

The PCB may include bends and/or folds (although the general dimensions for the unbent/unfolded PCB described above may still hold). The PCB may include any number of bends and/or folds limited such that the final PCB geometry can be incorporated into the end cap 120 and that electronic components on the PCB do not lose functionality (e.g., if the electronic component is situated on a PCB bend such that the electronic component is bent beyond function). In one example, as shown in FIG. 31, the implant circuitry is situated on a folded PCB inside the end cap 120. In some variations, the PCB may layout components and leads in coordination with planned folding patterns. For example, implant circuitry 140 may be positioned outside a defined folding seam to facilitate easier folding. With respect to the topology of the electronic components on the PCB, bends or folds may not occur on electronic components that cannot be bent or folded. For example, an antenna region may be folded without affecting the functionality of an antenna, while a fold may damage the functionality of a capacitor, although a bend may have no effect on the functionality of the capacitor (depending on the angle of the bend). Preferably, the layout of the implant circuitry 140 on the PCB may be configured into defined regions. In particular, the layout of components may include flex regions with no or minimal electronic components or components compatible with flexing. For example, in a variation where the PCB includes a 90° angle bend, there may be a region with no electronic component placement at the point of bending, and/or overlapping the region of bending/flexing. Additionally, conductive traces may be oriented across defined folding seams to mitigate mechanical issues of the leads.

In some variations, the implant circuitry 140 may have a power source. The power source functions to provide power for circuitry operation, particularly electrode operation. Dependent on implementation, the power source may comprise one, or multiple, power sources, where each power source may be of the same, or different type. In some variations, the power source may be an internal power source, i.e., located on, or within the implant, preferably within the end cap 120. Additionally or alternatively, the power source may be an external power source; i.e., located external to the implant, either as a separate implant or outside of the body of the patient.

Internal power sources may be housed within the end cap 120 of the implant, but can alternatively be outside of the end cap (e.g., within the tubular section of the surgical nail). Examples of internal power sources may include any type of energy storing devices, such as an internal battery (e.g., rechargeable), or capacitor(s). The internal power source may be electrically coupled to the implant control circuitry, the implant receiver system, the set of electrodes 130, and/or any other desired system component. In many variations, the system may include an internal power source(s) for regular operation, and an external power source for charging of the internal power source(s).

External power sources may comprise a separate implant and/or a source external to the patient. The external power source may be directly (e.g., by wiring) or indirectly (e.g., by induction) coupled to the implant and the implant circuitry 140. In variations that include a directly coupled external power source, the system may further include wiring connected to the end cap circuitry that extends from the implant to the external power source. This may include a shunt containing the wiring traveling from the implant within the patient to a position on the skin of the patient, such that the wiring may be “plugged-in” to charge the internal power sources.

The implant circuitry 140 may include an implant receiver circuitry. The implant receiver circuitry may function to send and receive electromagnetic signals both for communication and to provide electricity for the electrode operation. The implant receiver circuitry may enable external communication. That is, the implant receiver circuitry may function to enable communication with the system (and system components), and external components. Additionally or alternatively, the implant receiver circuitry may enable charging or powering of electronic components on, or within, the rod-like orthopedic implant 110. That is, the implant receiver circuitry comprises circuitry that enables power signal exchange, which functions to enable wireless delivery of power and/or communication with the implant and implant components, wherein an external device transmits power and/or data to the implant. That is, through the implant receiver circuitry, the implant, and/or implant components, may be wirelessly charged and/or powered by an external component. Additionally or alternatively, the implant receiver circuitry may enable transmission of data to external components (e.g., external implant circuitry, and/or external computing devices).

The implant receiver circuitry may be at least partially embedded in the end cap 120, and electrically connected to the implant control circuitry. The implant receiver circuitry may include at least one antenna. In one variation, the antenna is at least partially embedded in the end cap 120 and enabled to send and receive communication and electric current. In another variation, the antenna is completely located within the end cap 120. Additionally or alternatively, the implant receiver circuitry may include at least one antenna within (e.g., situated within the inner cavity of a tubular shaft), or along the surgical nail. This implant receiver circuitry may also have circuitry connecting it to electrical components within end cap 120.

The implant receiver circuitry may include one or more transmitter and/or receiver elements. Multiple transmitter and/or receive elements may be used. In one variation, these may be oriented in different directions to facilitate wireless coupling in different directions. In one variation, the implant receiver circuitry includes inductive coil(s) for coupling with a complimentary inductive coil of another device. In another variation, the implant receiver circuitry includes one or more RF (Radio Frequency) antenna(s), ultrasonic transducer(s), and/or other wireless power/data transmission elements.

One or more portions of the implant receiver circuitry may be wireless. Alternative implementations may use wired or direct communication. In some variations, data can be communicated through the wirelessly transmitted power signal, thereby enabling simultaneous (or near simultaneous) power and data transfer. For example, a high frequency data signal could be transmitted on top of a lower frequency power signal. The data signal could be decoded or read during conditioning of the received power signal. Data may include various commands relating to operational state directives, stimulation settings, diagnostics settings, communication settings, and/or other suitable commands. Data from the implant receiver circuitry is preferably implant operating data that may include current settings, diagnostics results, monitoring data, stimulation logs, power status, and/or other information. In some variations, data from the implant receiver circuitry may be held in local memory (e.g., as part of the implant control circuitry) until successful transfer to external components.

In some variations external components may transfer power and/or data to the implant receiver circuitry using a first dedicated set of tuned antennas. The implant receiver circuitry may transfer data to external components through a second, distinct set of tuned antennas; that is, the implant receiver circuitry may have a set of “sending” antennas and a set of “receiving” antennas. In another variation, external components may transfer data and/or power using a set of tuned antennas and the implant receiver circuitry may transfer data to external components through the same set of tuned antennas using, for example, load modulation.

In some variations, the power transmission can be modulated according to the power received by the implant receiver circuitry. For example, if the power supplied is not enough, external components may be instructed to adjust transmission (e.g., increasing transmission magnitude). In another variation, the data is communicated to external components wherein a doctor or processing unit may determine if any changes should be made to the electrical stimulation.

In one example implementation, the implant receiver circuitry comprises a receiver coil (e.g., a tuned air core planar receiver coil); rectifying circuitry; voltage regulator and an implantable transmitter coil. In this example implementation, the receiver coil and an external transmitter coil may form an inductive link where oscillating electric current within the external transmitter coil induces a potential over the tuned receiver coil through inductive coupling. Alternatively, depending on the transmitter type, the implant receiver circuitry may include receivers suitable for receiving RF irradiation, waves generated by an ultrasonic transducer and/or IR. In some variations, AC current in the receiver coil can be converted into DC current using rectifying circuitry. The voltage of the received signal may also be regulated using a voltage regulator. In one embodiment, capacitor(s) may store energy received through a wireless link and use it to meet the power consumption of the system circuitry. In one embodiment, the rectifying circuit may also function as an envelope detector, and the envelope of AC signals transmitted through the wireless link may be used to control the state of one or more of the system components, either directly or indirectly, via the implant control circuitry. In various embodiments, load modulation may be used to send data through the wireless link (e.g., from implant to external components).

The system may include implant control circuitry. The implant control circuitry functions to activate/deactivate, and control the implant circuitry 140. The implant control circuitry may be at least partially embedded in the end cap 120 and electrically connected to the set of electrodes 130, and to other “controllable” components (e.g., implant receiver circuitry, sensors, battery, etc.). In some variations, the implant control circuitry may be an external component (e.g., a computer) that communicates with the orthopedic implant via the implant receiver circuitry.

The implant control circuitry may also enable operating modes for the orthopedic implant. These operating modes may be implementation specific. Examples of different types of operating modes may comprise: different types of electrode activation, both for sensory functionality and for providing tissue stimulation; operation of sensor components; application of externally provided stimulation activity (e.g., doctor prescribed); and/or other types of operation. Dependent on implementation, one or multiple operating modes may be active at one time. In this manner, the implant control circuitry may function to provide real time stimulation and monitoring of tissue, wherein complex patterns of activation and deactivation of electrodes and operating modes may be implemented for relatively precise tissue stimulation.

The implant control circuitry may provide an electrode stimulation operating mode. Electrode stimulation mode may function to provide tissue stimulation to help modify tissue growth (e.g., increase/decrease bone growth/reduction). During the electrode stimulation mode, the implant control circuitry may activate one, multiple, or all electrodes from the set of electrodes 130 to provide electric stimulation (i.e., activates a subset of electrodes from the set of electrodes). As part of the electrode stimulation mode, the implant control circuitry may set the polarity of each electrode. The polarity may include the charge (e.g., positive, negative, or neutral/inactive), and the charge magnitude. Thus, the implant control circuitry may enable each stimulation site to function as a current source or sink, enabling current to travel through tissue in proximity to the stimulation. In one example of the electrode stimulation mode, electrodes along the shaft of the surgical nail are activated as current sink to provide tissue growth stimulation around the nail. A current source may be activated at, or near the head to direct the current flow along the surgical nail.

In another example of stimulation operating modes, the implant control circuitry may enable “color-based” excitation (i.e., color-based stimulation operating mode). More generally, the stimulation operation modes may be specified based on regional designations. That is, through the implant control circuitry, a user may provide an input indicating one or more regional designations to direct stimulation (e.g., red stimulation). In one implementation, where color coincides with lengthwise positioning of the electrodes, a “red” stimulation may coincide with providing electrode stimulation at, or near, the tail of the surgical nail. As discussed above, in some cases, the regional designations may be apparent when the subject undergoes medical imaging.

The implant control circuitry may additionally provide an impedance measuring operating mode. The impedance measuring operating mode may function to determine the type (and amount) of tissue between a pair (or more) of electrodes, by measuring the impedance between the pair of electrodes. During the impedance measuring operating mode, the implant control circuitry may activate a designated subset of electrodes as current sources and activate a designated subset of electrodes as current sinks. Furthermore, the implant control circuitry may measure the tissue impedance of the current as it travels through tissue from the current source to the current sink. In one example for impedance measurement. One electrode near the tip of the shaft of a surgical nail may be activated as a current source, and an electrode directly opposite of the current source is activated as a current sink. The implant control circuitry may then measure the impedance of the tissue at that “height” along the surgical nail between the two electrodes. From known impedance tissue measurements, and other impedance measurements of tissue, the tissue impedance may then be used to determine the type and quantity of tissue between the two electrodes.

The implant control circuitry may further include configuration to control stimulation based on measured impedance. For example, an impedance profile characterizing the spatial impedance properties in proximity to the surgical nail can be generated through the impedance measuring operating mode. Then a stimulation profile may be generated or determined based on the impedance profile and then used in driving stimulation across the set of electrodes. The stimulation profile may additionally be determined based on set configuration. For example, a surgical nail may be configured to limit stimulation to a sub-region (and thereby only a subset of the set of electrodes) so dynamic stimulation may be limited to a certain region along the length of the surgical nail.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

The system functionality may be implemented at least in part as through the implant control circuitry and/or other internal or external control systems configured to receive a computer-readable medium storing computer-readable instructions. In one variation, instructions may be executed by the implant control circuitry. In another variation, instructions may be executed by an external computing system that is in communication with the implant device and which updates the implant control circuitry to alter operation of the implant device. In another device, instructions configured within a combination of the implant control circuitry and an external computing system. Other systems and methods of the embodiment can be embodied and/or implemented at least in part as a device configured to receive a computer-readable medium storing computer-readable instructions that can be communicated to and/or executed by the system(s) described herein. The instructions can be executed by the internal control circuitry 150 and/or other computer-executable components integrated with apparatuses and networks of the type described herein. The computer-readable medium can be stored on any suitable computer readable media such as RAMs, ROMs, flash memory, EEPROMs, optical devices (CD or DVD), hard drives, floppy drives, or any suitable device, wherein the storage medium may be located internally (e.g., integrated with the control circuitry) and/or externally as part of some general storage device(s) or external computing system in communication with the implantable system. The computer-executable component can be a control circuitry 150 and/or a processor but any suitable dedicated hardware device can (alternatively or additionally) execute the instructions.

In one variation, a system comprising of one or more computer-readable mediums storing instructions that, when executed by one or more computer processors or circuitry of the implant control circuitry, cause the processors/circuitry to perform operations comprising those of the system operating modes or methods described herein such as: activating an electrode stimulation mode; and activating an impedance measuring mode; increasing electrical stimulation; decreasing electrical stimulation; augmenting the electrode stimulation mode in response to measured impedance and/or other sensed conditions.

FIG. 31 is an exemplary computer architecture diagram of one implementation of the implant control circuitry. In some implementations, the implant control circuitry is implemented in a plurality of devices in communication over a communication channel and/or network (e.g., the control circuitry within the surgical implant and an external computer processor). In some implementations, the elements of the control circuitry 150 are implemented in separate computing devices. In some implementations, two or more of the implant control circuitry elements are implemented in same devices. The implant control circuitry and portions of the control circuitry may be integrated into a computing device or system that can serve as or within the system.

The communication channel 1001 interfaces with the processors 1002A-1002N, the memory (e.g., a random access memory (RAM)) 1003, a read only memory (ROM) 1004, a processor-readable storage medium 1005, a display device (e.g., external monitor) 1006, a user input device 1007, and a network device 1008. As shown, the computer infrastructure may be used in connecting electrodes 1101, an implant receiver circuitry 1102, a power source, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid state drive, a ROM, an EEPROM, an electronic circuit, a semiconductor memory device, and the like. The processor-readable storage medium 1005 can include an operating system, software programs, device drivers, and/or other suitable sub-systems or software.

As a person skilled in the art will recognize from the previous detailed description and from the figures and claims, modifications and changes can be made to the embodiments of the invention without departing from the scope of this invention as defined in the following claims.

We claim:

1. A system for an electrically stimulating orthopedic implant comprising:
a rod-like orthopedic implant, comprising the shaft of the orthopedic implant, with distinct electrode sites situated along the implant body;
an end cap, comprising the head of the orthopedic implant, connected and fixed in place to one end of the rod-like orthopedic implant;
a set of electrodes, individually controllable, wherein each electrode:
includes a distinct stimulation site, comprising an active exposed segment of the electrode situated on an electrode site,
is conductively coupled to an implant control circuitry, and
is conductively isolated from all other electrodes in the set of electrodes; and
implant circuitry, situated within the end cap, comprising:
implant receiver circuitry effective to convert an electromagnetic field to an electrical current and
implant control circuitry configured to control current flow of the set of electrodes.

2. The system of claim 1, wherein the implant receiver circuitry comprises an antenna system, at least partially embedded in the end cap of the rod-like orthopedic implant and enabled to send and receive communication and electric current.

3. The system of claim 2, wherein the implant circuitry is situated on a folded PCB inside the end cap.

4. The system of claim 3, wherein the end cap is connected to the rod-like orthopedic implant through a conductive connector that includes an electrical conduit that connects the implant circuitry and the set of electrodes.

5. The system of claim 4, wherein the end cap is hermetically sealed.

6. The system of claim 2, wherein through the implant receiver circuitry the implant may be wirelessly charged by an external component.

7. The system of claim 2, wherein the rod-like orthopedic implant is primarily composed of titanium and the end cap is primarily composed of PEEK.

8. The system of claim 7, wherein the orthopedic implant comprises a surgical nail.

9. The system of claim 8, wherein the surgical nail shaft comprises an at least partially solid segment.

10. The system of claim 9, wherein the electrode sites are etched onto the surface of the surgical nail.

11. The System of claim 8, wherein the surgical nail comprises an at least partially tubular segment defining an internal cavity within the surgical nail.

12. The system of claim 11, wherein electrode circuitry travels through the interior cavity of the implant body and connects to the electrode stimulation sites through holes in the shaft of the surgical nail.

13. The system of claim 11, wherein the surgical nail comprises an at least partially open segment, such that along the open segment of the surgical nail, the interior surface and the external surface of the surgical nail form a continuous surface.

14. The system of claim 8, wherein electrode stimulation sites are positioned along the length of the surgical nail.

15. The system of claim 14, wherein electrode stimulation sites comprise rings around the shaft of the surgical nail.

16. The system of claim 14, wherein the implant control circuitry provides an electrode stimulation mode, wherein during the electrode stimulation mode, the implant control circuitry activates a subset of electrodes from the set of electrodes to provide electric stimulation.

17. The system of claim 16, wherein during an electrode stimulation mode, the implant control circuitry provides color-based excitation, wherein a color coincides to a lengthwise position along the shaft of the surgical nail.

18. The system of claim 8, wherein the system further includes insulation, wherein insulation is situated on each electrode site thereby conductively isolating the electrode stimulation site from the surgical nail.

19. The system of claim 8, wherein the implant control circuitry provides an impedance measuring operating mode, such that in an impedance measuring operating mode, the impedance between a pair of electrodes is measured.
